(19) United States
(12) Patent Application Publication
(10) Pub. No.: US 2005/0195477 A1
(43) Pub. Date: Sep. 8, 2005

(54) Method and Apparatus for the Suppression of Pinning in Stereoscopic Display Systems

(75) Inventor: Cary Dikel Kornfeld, Mountain View, CA (US)

Correspondence Address: CARY DIKEL KORNFIELD, 468 SIERRA VISTA #7, MOUNTAIN VIEW, CA, 94043, US

(73) Assignee: Cary Dikel Kornfeld, Mountain View, CA (US)

(21) Appl. No.: 10/708,419

(22) Filed: Mar. 2, 2004

(51) Int. Cl. A61B 3/08; G02B 27/22

(52) U.S. Cl. 359/462; 351/201

(57) ABSTRACT

The perception of depth in human vision results from a variety of visual information. Spatial interpretation depends on a consistent interpretation of all types of visual information (e.g. retinal disparity, motion parallax, occlusion, etc.). Apparatus and methods are presented that suppress conflicts among conflicting visual information. In particular, conflicts between occlusion caused by partially occluded stereoscopic displayed objects and retinal disparity are suppressed by use of a variety of means.

### BACKGROUND OF INVENTION

References Cited
Vision Research, Vol. 14, no. 10, p. 975-82 1974
Vision Research, Vol. 28, no. 4, p. 555-62, 1988
Neural Networks, Vol. 6, no. 4, p. 463-83, 1993

### SUMMARY OF INVENTION

The present invention relates to the augmentation of stereoscopic display systems to suppress undesired visual artifacts.

Stereoscopic image display systems rely on the presentation of slightly different views of a scene to each eye of the viewer. Objects within these separate images are laterally displaced such that each eye sees the same object at somewhat different locations. The discrepancy between perceived location creates a condition known as retinal disparity. The human visual system infers depth by means of this discrepancy and if all such objects within the field of view exhibit consistent stereoscopic clues, the observer perceives a sense of depth and thus a stereoscopic illusion. A variety of stereoscopic display systems have been described in the patent literature.

The human visual system relies on a variety of visual clues as it determines depth from the visual images it sees. Depth can be perceived by means of relative motion, relative size, increased detail in objects closer to the viewer, etc. It is well known that some depth clues are more powerful than others. For example, persons with monocular vision can still perceived depth by means of relative motion and by means of observing some objects to obscure others in the field of view. Those objects that obscure others are interpreted as being closer than those objects they obscure, for example.

Human vision relies on a complex and little understood process commonly known as “scene interpretation” as it brings order to visual perception. Such processes include segmentation, color perception, edge detection, etc. If a viewer is presented with conflicting visual clues the normal human response is to restart the process of “scene interpretation” to determine if a more plausible interpretation of the visual field can be made.

Consistency of visual clues is extremely important in the creation of stereoscopic imaging. If conflicts occur the sensation of depth is lost and the viewer perceives double vision. The stereoscopically encoded objects are no longer fused and interpreted to appear at various points in space, but are seen as two distinct views that is confusing and uncomfortable for the viewer.

The movement of stereoscopic objects off of the stereoscopic display causes a common problem encountered in stereoscopic images. Objects perceived to be floating in front of the display begin to disappear “behind” the margin of the display device. These objects are occluded by an visual percept that has previously been interpreted as behind the stereoscopic object. Occlusion is a more powerful stereoscopic visual clue than is retinal disparity. When these objects move off screen a conflict in visual clues is perceived causing a loss of the illusion of stereoscopic depth. This condition is commonly referred to as stereoscopic “pinning”.

Pinning can be controlled when creating stereoscopic movies by insuring that objects perceived as in front of the surface of the display (which is known as the zero parallax plane) are never moved off screen until they have receded to a position that is interpreted as behind the edge of the screen. Since the movie is carefully shot, pinning can be avoided by careful attention to the placement and movement of actors and objects within the scene.

Accordingly, several objects and advantages of this invention over existing methods and the teaching of the prior art include the object to provide means for suppressing “pinning” during the presentation of stereoscopic images, videos, motion pictures and computer generated and or presented stereoscopic material.

### BRIEF DESCRIPTION OF DRAWINGS

FIG. 1 depicts the spectral sensitivities of the three color sensing cones in the retinal mosaic. These are commonly termed Short, Medium and Long Wavelength Sensor and are abbreviated as SWS, MWS and LWS. Label 102 depicts the Long wavelength cone sensitivity curve. Label 104 designates the Medium wavelength cone sensitivity curve. Label 106 designates the Short wavelength cone sensitivity curve. Each cone sensor type can received a broad spectrum of light. Color is derived by comparing the signaling strength from neighboring cones. The Medium and Long Wavelength cone sensitivities overlap broadly whereas the Short Wavelength Sensor type cone has a narrow spectral range that does not broadly overlap the sensitivities of the MWS and LWS cones. As depicted in FIG. 1, light whose frequency is at or near 450 nanometers is primarily sensed by the SWS cones.

FIG. 2 depicts the relative distribution of short, medium and long wavelength sensing cones. The figure consists of two micrographs of the same region of the retinal mosaic. The, labeled 202 includes the macular pigment. The second, labeled 204 depicts the same region without the macular pigment. The cones are more clearly seen in the region labeled 204. The Long wavelength cones are depicted by label 206. Label 208 depicts the Medium wavelength cones whereas label 210 designates the Short wavelength cones. There are approximately sixteen times more medium and long wavelength cones than short wavelength cones as is indicated in this micrograph.

FIG. 3 depicts the central region of the fovea: the region of highest visual acuity. The area designed as 302 depicts the fovea region whereas label 304 indicates the central fovea. In this region of the retina the cone cells are smaller and more tightly packed than in the periphery of the retina. Moreover there are no short wavelength sensors in the central fovea.

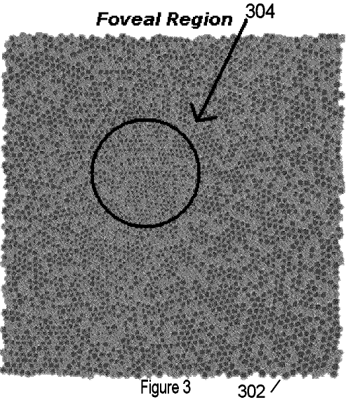

FIG. 4 shows a stylized representation of the apparatus described in this patent. Element 402 is the stereoscopic display device including border element 404 and display element 406. The viewer's right and left eyes are depicted as elements 408 and 410, respectively. The anti-pinning border is defined by the region bounded by elements 412 and 414.

### DETAILED DESCRIPTION

Although the following detailed description contains many specifics for the purposes of illustration, anyone of ordinary skill in the art will appreciate that many variations and alterations to the following details are within the scope of the invention. Accordingly, the following preferred embodiment of the invention is set forth without any loss of generality to, and without imposing limitations upon, the claimed invention.

The present invention accordingly has an objective to provide a variety of novel means for the creation of apparatus for presentation of stereoscopic material.

A preferred embodiment of this invention employs a perimeter around the displayed stereoscopic image that serves to provide ambiguous depth clues. Such ambiguity reduces or eliminates a well known stereoscopic artifact that has limited the use of stereoscopic imaging. This artifact is known in the art as “pinning”. It occurs whenever a stereoscopic object is perceived to be positioned as if floating in front of the display surface. If that object is translated so that it either touches or begins to move off the edge of the display, the visual system observes two conflicting visual clues: an object interpreted as being in front of the display is occluded by the edge of the display (which is perceived as behind the object). Since occlusion is a more powerful visual depth clue than is retinal disparity, the illusion of depth is destroyed and the viewer no longer perceives a stereoscopic image.

The problem of pinning occurs in all real time stereoscopic imaging systems. Those skilled in the art of creating stereoscopic content knowingly create content such that forward objects never intersect the edge of the display so as to prevent pinning from occurring.

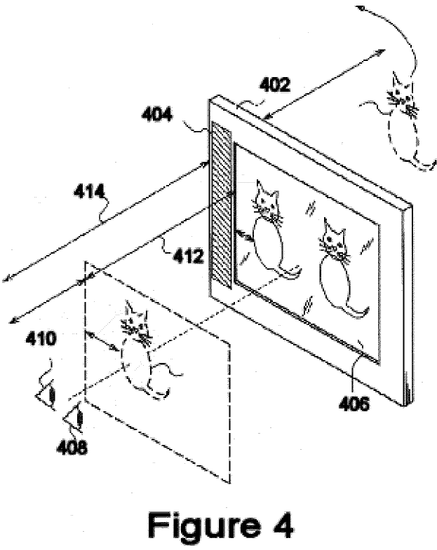

The apparatus described in this invention and shown in FIG. 4 employs a luminous blue border along the periphery of the displayed image. The use of blue light is intentional because of a variety of factors related to the human visual system.

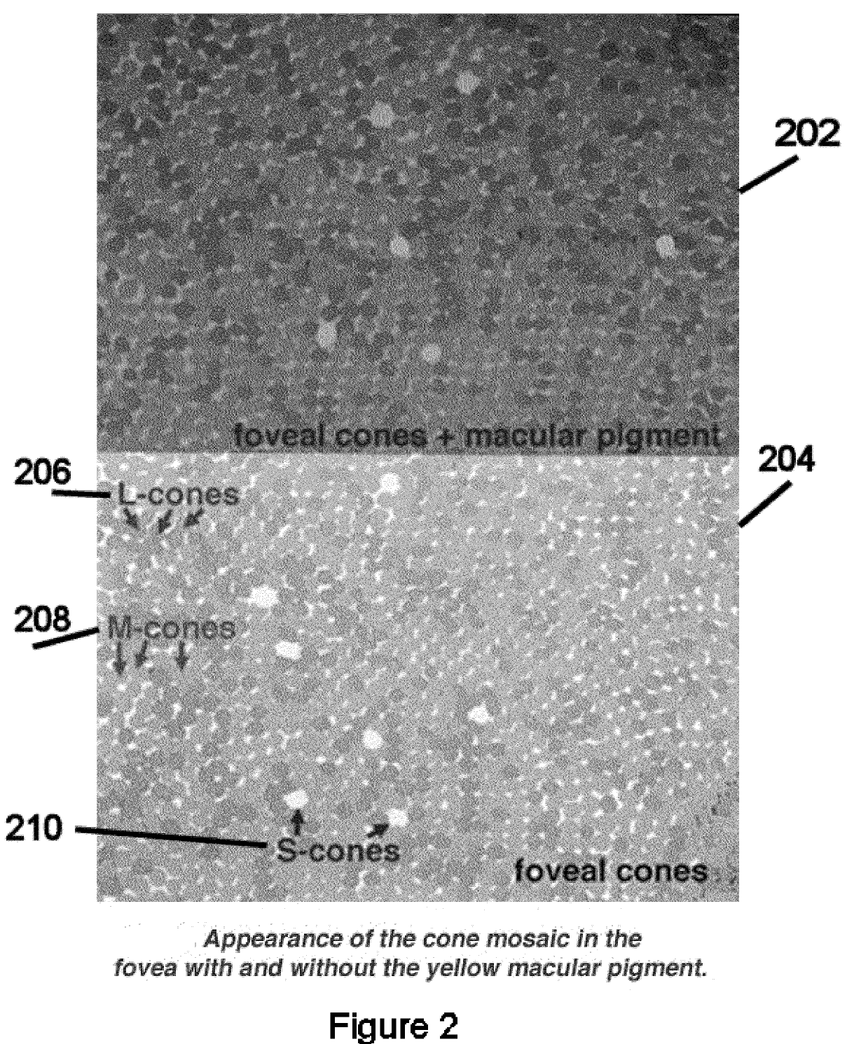

It is well known to those skilled in human vision that the lens of the human eye is a simple lens that is achromatic. That is, the lens focuses light of different wavelengths (colors) at various position in space. Light of deep blue color is always focused at a point that is substantially in front of the retina and is thus always perceived as out of focus. Moreover, blue light is primarily sensed by one of three color receptor cells of the retina which are called SWS (Short Wavelength Sensor) cones. The region of the human retina which has the highest spatial acuity is located in a region known as the fovea region of the retina. It is well known that SWS cones are not present in the central region of the fovea. SWS cones begin to occur in the peripheral region of the fovea and are significantly less numerous, as is shown in FIG. 2, than the other two types of color receptive cells (the MWS and LWS cones). Various studies report that there are as few as one sixteenth as many SWS cones in the retina as other color sensors. The perception of blue light and in particular the use of a luminous blue border around the stereoscopic image provides ambiguous visual depth clues that inhibit and reduce the problem of pinning. Ideally, the luminous blue border should generate light that is only perceived by the SWS cone sensors (a wavelength of approximately 450 nanometers or shorter).

The illusion of depth created by stereoscopic imaging is dependent on presenting slightly different images to each eye. Elements within each image are displaced laterally in a manner that is proportional to their perceived distance to the viewer. It is clear that the border of the stereoscopic displayed image can become a factor that can inhibit the perception of depth even though consistent stereoscopic cues (retinal disparity) are provided throughout the displayed image.

Moreover, the luminous deep blue border along the edge of the display surface can be constructed so that the edge of the displayed image merges into the bordering region such that the intensity of the edge of the displayed image is subtly merged to deep blue, thus decreasing the discontinuity between the border of the displayed stereoscopic image and the image itself.

The apparatus now disclosed and described can be further augmented to enhance the stereoscopic viewing experience by employing a luminous blue perimeter bounding the edge of the stereoscopic display area as shown in FIG. 4.

It is well known within the art that the human visual system employs a variety of visual data in order to perceive a sense of depth. These include relative size, relative motion, retinal disparity and object occlusion. Object occlusion is a more powerful visual clue than is retinal disparity.

It also well known that stereoscopic objects made to appear as if in front of the display surface can cause a conflict in visual clues if said object(s) move to or off the field of view. The object appears to have disappeared behind the edge of the display and thus be occluded by an object that was initially perceived as behind said object. When this occurs the perception of depth is lost. The two displayed images are no longer merged so as to perceive depth but now appear as two slightly displayed images. The illusion of depth is lost. “Pinning” is a term that is used to refer to the creation of this condition in stereoscopic display systems.

It has been discovered that “pinning” can be suppressed and/or eliminated by means of providing a luminous deep blue border around the periphery of the stereoscopic display area.

Some aspects of human vision, although studied, are not well understood. But a rational argument can be made as to why this effect occurs. For example, the lens of the eye is a simple lens subject to chromatic distortion. Different wavelengths (colors) of light come to focus at different distances from the lens and the retina. Some colors (e.g. red) come to focus at a position that is somewhat behind the surface of the retina. Conversely, other colors, such as blue (short wavelength light) come to focus substantially in front of the retina. The human visual system is not capable of focusing blue light onto the retina of the eye.

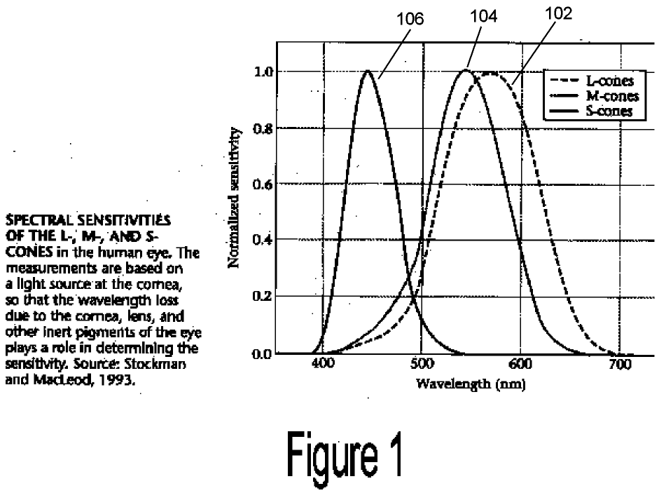

The perception of color in the human visual system is not well understood. A number of retinal sensing cells (cones and rods) have been identified. The frequency response and spatial distribution of these cells has been studied in great detail. There are three types of cone or color sensing cells within the eye which are typically called Long Wavelength, Medium Wavelength and Short Wavelength Sensors (L, M and S cones). As shown in FIG. 1, the frequency response of the L and M cones is broad whereas the S cones have a narrow frequency response. And although there is considerable overlap in the sensitivities of the L and M cones there is relatively little such overlap in the frequency response of the S cones.

It is well known that there are no S cones in the most sensitive region of the retina which is called the fovea. Whereas most regions of the retina consist of a mixture of rod and cone sensing cells, the fovea only contains cone type cells and only those which are of the L and M types. S type cones are only detected in the periphery of the fovea region and their spatial distribution is much lower than either the L or M cones. It is estimated that there are less than one sixteenth as many S cones as either L or M cones in the retina.

Since short wavelength sensing cells (S-cones) do not occur in the central region of the fovea, it is well known that it is visually impossible to attain focus on an object emitting only shortwave length (e.g. blue only). The elements of the image are defocused by virtue of the achromatic characteristics of the lens of the eye and also by the absence of appropriate sensor cells within the prime focus region of the retina.

The combination of defocused short wavelength (blue) light and lack of S cones in the area of highest visual acuity results considerably lower spatial and temporal sensitivity of the human eye to blue light. Blue objects must be substantially larger in size to be perceived clearly by the eye. These factors appear to make difficult, if not impossible to derive depth information from purely blue elements.

A luminous blue border around a stereoscopic image creates a zone of depth ambiguity about the location of the screen on which the image is displayed image. This ambiguity can be employed to suppress and possibly inhibit stereoscopic pinning and is employed for this purpose in this patent.

FIG. 1 teaches that there is little overlap in spectral response between S- and either L- or M-cones at wavelength at 450 nm or less. As such, the luminous border described should emit light at approximately this or shorter wavelengths.

1. An apparatus for preventing loss of depth perception by a viewer due to depth pinning, said apparatus comprising:
a) a stereoscopic image display for stereoscopically generating an object perceived by said viewer at a perceived depth;
b) a periphery around said stereoscopic image display;
c) an anti-pinning element positioned in said periphery for projecting radiation comprising a blue wavelength to said viewer, thereby preventing said viewer from depth pinning at said stereoscopic image display.

2. The apparatus of claim 1, wherein said anti-pinning element is an active emitter of said radiation.

3. The apparatus of claim 2, wherein said active emitter is a light source mounted in said periphery

4. The apparatus of claim 1, wherein said anti-pinning element is coextensive with said periphery.

5. The apparatus of claim 1, wherein said object is perceived by said viewer at a perceived depth within a field of view and said anti-pinning element is positioned at a location in said periphery where said object appears to said viewer to approach a border of said field of view.

6. The apparatus of claim 1, wherein said stereoscopic image display comprises at least one screen and stereoscopically generates said object from elements projected on said at least one screen.

7. The apparatus of claim 1, wherein said stereoscopic image display augmented with an anti-pinning element positioned in said periphery for projecting radiation comprising a blue wavelength to said viewer is activated only when objects approach the border of the viewer's field of view.

8. The apparatus of claim 1, wherein said stereoscopic image display augmented with an anti-pinning element positioned in said periphery for projecting radiation comprising a blue wavelength to said viewer vary intensity as objects approach the border of the viewer's field of view.

9. The apparatus of claim 6, wherein said image display comprises a multiplicity of display screens having multiple interfaces, and additional anti-pinning elements positioned at said interfaces for projecting radiation comprising said blue wavelength to said viewer.

10. The apparatus of claim 1, wherein said blue wavelength comprises wavelengths inducing depth ambiguity in said viewer, thereby preventing said viewer from depth pinning.

11. A method for preventing loss of depth perception by a viewer due to depth pinning, said method comprising:
a} stereoscopically generating an object on a stereoscopic image display, such that said object is perceived by said viewer at a perceived depth;
b) providing a periphery around said stereoscopic image display;
c) positioning an anti-pinning element in said periphery for projecting radiation comprising a blue wavelength to said viewer, thereby preventing said viewer from depth pinning at said stereoscopic image display.

12. The method of claim 11, wherein said step of stereoscopically generating said object employs a stereoscopic technique selected from the group consisting of:
a) Time multiplexed presentation of left and right image in conjunction with apparatus synchronized with said presentation so that the left eye views the left image when said left image is displayed and right eye views the right image when said right image is displayed;
b) Spatially multiplex presentation of left and right image combined with a viewing apparatus that enables the right eye to see the right image and the left eye to see the left image;
c) Simultaneous presentation of both left and right images such that the left eye sees the left image and the right eye sees the right image by virtue of the cross-polarization of the light and simultaneous use of polarized viewing device such that each eye sees one of the two cross polarized images;
d) simultaneous presentation of both left and right images such that the left eye sees the left image and the right eye sees only the right image by virtue of color encoding differences between each image and simultaneous use of color matched viewing device such that each eye sees one of the two color encoded images (e.g. anaglyph);
e) Time multiplexed presentation of left and right image in conjunction with a polarizing device interposed between the display image and the viewer, capable of switching the polarization of the light passing through and thus providing time multiplexed presentation of left image with one polarization and the right image with a crossed polarization combined with a viewing apparatus that enables the right eye to see the right image and the left eye to see the left image;
f) Stereoscopic images created by use of intensity attenuating viewing apparatus such that one eye sees a darkened image and the other eye sees a brightened image thus causing the visual system to take longer to process the darkened image than the brightened image and thus matching two precepts at slightly different points in time (e.g. Purflich effect);
g.) Stereoscopic images created by using a multiplicity of stacked display surfaces that vary slightly in distance to the viewer causing said viewer to perceived depth by virtue of the visual system seeing elements on said multiplicity of stacked display surfaces as positioned at different distances;
h) Stereoscopic display devices designed such that each eye is presented with a different image such that the left eye sees a left image and the right eye sees a right image, said display device created by use of optical components that direct each image to the eye that is intended to see that image (e.g. virtual reality goggles);
i) Any display device capable of inducing a stereoscopic illusion.

13. The method of claim 11 wherein said viewer perceives said object within a field of view having a border, and said anti-pinning element is positioned such that it appears to said viewer near said border.

14. The method of claim 11 wherein said anti-pinning element emits radiation comprising said blue wavelength.

15. The method of claim 11, wherein said anti-pinning element emits radiation comprising said blue wavelength are only employed when objects approach the border of the viewer's field of view.

16. The method of claim 11, wherein said anti-pinning element emits radiation comprising blue wavelength of light vary in intensity as objects approach the border of the viewer's field of view

17. A method for preventing loss of depth perception by a viewer due to depth pinning, said method comprising:
a) stereoscopic generating an object being perceived by said viewer at a perceived depth within a field of view having a border;
b) positioning an anti-pinning element such that said viewer perceives said anti-pinning element near a border of said field of view, thereby preventing said viewer from depth pinning.

18. The method of claim 17 wherein said step of stereoscopically generating said object employs a stereoscopic technique selected from the group consisting of:
a) Time multiplexed presentation of left and right image in conjunction with apparatus synchronized with said presentation so that the left eye views the left image when said left image is displayed and right eye views the right image when said right image is displayed;
b) Spatially multiplex presentation of left and right image combined with a viewing apparatus that enables the right eye to see the right image and the left eye to see the left image
c) Simultaneous presentation of both left and right images such that the left eye sees the left image and the right eye sees the right image by virtue of the cross-polarization of the light and simultaneous use of polarized viewing device such that each eye sees one of the two cross polarized images;
d) simultaneous presentation of both left and right images such that the left eye sees the left image and the right eye sees only the right image by virtue of color encoding differences between each image and simultaneous use of color matched viewing device such that each eye sees one of the two color encoded images (e.g. anaglyph);
e) Time multiplexed presentation of left and right image in conjunction with a polarizing device interposed between the display image and the viewer, capable of switching the polarization of the light passing through and thus providing time multiplexed presentation of left image with one polarization and the right image with a crossed polarization combined with a viewing apparatus that enables the right eye to see the right image and the left eye to see the left image;
f) Stereoscopic images created by use of intensity attenuating viewing apparatus such that one eye sees a darkened image and the other eye sees a brightened image thus causing the visual system to take longer to process the darkened image than the brightened image and thus matching two precepts at slightly different points in time (e.g. Purflich effect);
g.) Stereoscopic images created by using a multiplicity of stacked display surfaces that vary slightly in distance to the viewer causing said viewer to perceived depth by virtue of the visual system seeing elements on said multiplicity of stacked display surfaces as positioned at different distances;
h) Stereoscopic display devices designed such that each eye is presented with a different image such that the left eye sees a left image and the right eye sees a right image, said display device created by use of optical components that direct each image to the eye that is intended to see that image (e.g. virtual reality goggles);
i) Any display device capable of inducing a stereoscopic illusion.

19. The method of claim 17 wherein said object is projected on a stereoscopic image display

20. The method of claim 19 wherein said stereoscopic image display comprises a periphery and said anti-pinning element is positioned in said periphery.

21. The method of claim 17 where n anti-pinning methods are only employed when objects approach the border of the viewer's field of view.

22. The method of claim 17, wherein said anti-pinning methods vary in intensity as objects approach the border of the viewer's field of view.
